(19) United States
(12) Patent Application Publication
(10) Pub. No.: US 2026/0102180 A1
(43) Pub. Date: Apr. 16, 2026

(54) DEVICES, SYSTEMS, AND METHODS FOR MOVING TISSUE DURING A PROCEDURE

(71) Applicants: BOSTON SCIENTIFIC SCIMED, INC., Maple Grove, MN (US); BOSTON SCIENTIFIC MEDICAL DEVICE LIMITED, Galway (IE)

(72) Inventors: Deepak Kumar Sharma, Muzaffarnagar (IN); Austin Grant Johnson, Worcester, MA (US); Scott Edward Corbeil, Litchfield, NH (US); Nachiket Gole, Pune (IN); Sharath Kumar G., Kanakapura (IN)

(73) Assignees: BOSTON SCIENTIFIC SCIMED, INC., Maple Grove, MN (US); BOSTON SCIENTIFIC MEDICAL DEVICE LIMITED, Galway (IE)

(21) Appl. No.: 19/354,622

(22) Filed: Oct. 9, 2025

Related U.S. Application Data
(60) Provisional application No. 63/706,286, filed on Oct. 11, 2024.

(51) Int. Cl. A61B 17/32; A61B 17/00; A61B 17/3209

(52) U.S. Cl. CPC A61B 17/320016; A61B 17/3209; A61B 2017/00818; A61B 2017/320056

(57) ABSTRACT

A tissue moving device and apparatus with an expansion section including a plurality of elongated expansion elements shiftable from a delivery configuration to an expanded configuration. The elongated expansion elements may be separately and independently shiftable to allow various expansion configurations. The elongated expansion elements may have an end coupled to a support which is segmented into a plurality of support segments, each independently movable to allow independent shifting of the associated elongated expansion element with respect to the other elongated expansion elements. The expansion section may be mounted over a shaft, such as a shaft of a cutting device, or a shaft with a lumen through which a cutting device is axially translatable.

### CROSS-REFERENCE TO RELATED APPLICATIONS

This application claims the benefit of priority under 35 U.S.C. § 119 to U.S. Provisional Application No. 63/706,286, filed Oct. 11, 2024, the entire disclosure of which is hereby incorporated by reference herein for all purposes.

### FIELD

The present disclosure relates generally to the field of medical devices, systems, and methods for moving anatomical tissue, such as separating and/or lifting anatomical tissue away from other anatomical tissue, such as underlying anatomical tissue being cut, dissected, resected, etc., during a medical procedure.

### BACKGROUND

Various devices, assemblies, systems, methods, techniques, etc., exist for cutting, dissecting, resecting, excising, etc., anatomical structures (e.g., biological tissue, including lesions, etc.). One endoscopic procedure is third space endoscopy (also known as submucosal endoscopy), which may access deeper layers of tissue within the body (e.g., within the gastrointestinal (GI) tract), such as between structurally differentiated layers of tissue. For instance, the submucosal space may be accessed with an endoscope, without compromising the integrity of the overlying mucosa. Typically, a fluid (e.g., saline) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A cutting knife may then be used to cut through the submucosa. During endoluminal surgery procedures, the medical professional typically holds and manipulates the medical scope with one hand (e.g., the left hand) and manipulates the insertion shaft/tube of the endoscope with the other hand (e.g., the right hand) to get the scope in the desired target position. Once the scope is in the desired location, a technician hands over the relevant accessories (snare, knife, radiofrequency (RF) knife, scissors, etc.) to the medical professional who then introduces the accessories into the scope. The medical professional positions the accessory by torquing the scope shaft, the accessory, and/or the scope handle. The technician typically investigates preparing and/or actuating the accessory before introducing it into the scope, and injects saline or a specific medium into the accessory based on the medical professional's instruction and the procedure being performed. Presently, getting the accessory in the right position is a combination of scope handle manipulation, shaft manipulation, accessory manipulation, accessory torquing, etc., and, to complete the procedure/task, the accessory needs to be actuated. The resecting tool is extended through the scope (e.g., through a working channel of the scope), and a traction device is extended over the scope to the target site of the procedure. Since the traction device is attached to the endoscope, it is difficult to maintain continuous traction during endoscope movement. There remains a need for improved devices, methods, and systems for lifting tissue away as underlying tissue is being cut, such as to be able to visualize the cutting plane during resection. Solutions to these and other challenges in the art would be welcome, and itis with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a medical device is configured to move tissue. In some aspects, the medical device includes an expandable section having a proximal end and a distal end and extending along a longitudinal axis of the medical device; and one or more elongated expansion elements each having a proximal end coupled with respect to the proximal end of the expandable section and a distal end coupled with respect to the distal end of the expandable section. In some aspects, the proximal end and distal end of at least one of the one or more elongated expansion elements are movable with respect to each other to shift the at least one of the one or more elongated expansion elements between a delivery configuration extending along the longitudinal axis and an expanded configuration extending laterally away from the longitudinal axis to shift the expandable section between a delivery configuration and an expanded configuration.

In some aspects, the medical device further includes a proximal support along a proximal end of the expandable section, the proximal end of the at least one of the one or more elongated expansion elements coupled to the proximal support; and a distal support along a distal end of the expandable section, the distal end of the at least one of the one or more elongated expansion elements coupled to the distal support. In some aspects, the proximal support and the distal support are movable with respect to each other to shift the at least one of the one or more elongated expansion elements between the delivery configuration and the expanded configuration. In some aspects, the one or more elongated expansion elements include a plurality of elongated expansion elements extending longitudinally along and circumferentially around the expandable section. In some aspects, each of the plurality of elongated expansion elements has a distal end coupled to the distal support and a proximal end coupled to the proximal support; and at least one of the distal support or the proximal support is segmented circumferentially to define support segments which move longitudinally separately and independently of one another to shift the elongated expansion elements separately and independently of one another between the delivery configuration and the expanded configuration thereof.

In some aspects, the medical device further includes a control element operably coupled with one of the distal support or the proximal support to move the proximal support and the distal support with respect to each other. In some aspects, the control element has a proximal end extending proximal to the proximal end of the at least one of the one or more elongated expansion elements and to a control handle at a proximal end of the medical device. In some aspects, one of the proximal support or the distal support is annular defining a passage therethrough and movable with respect to a shaft extended through the passage. In some aspects, the other of the proximal support or the distal support is fixed with respect to the shaft. In some aspects, the shaft defines a lumen therethrough through which a cutting device is axially translatable with respect to the expandable section.

In some aspects, the expandable structure is operably associated with a shaft defining a lumen therethrough through which a cutting device is axially translatable with respect to the expandable section.

In accordance with various principles of the present disclosure, a system for separating tissue includes a tissue moving device having an expandable section having a proximal end and a distal end and extending along a longitudinal axis of the medical device, and one or more elongated expansion elements each having a proximal end coupled with respect to the proximal end of the expandable section and a distal end coupled with respect to the distal end of the expandable section; and a cutting device, the expandable section operably associated with the cutting device. In some aspects, the proximal end and distal end of at least one of the one or more elongated expansion elements are movable with respect to each other to shift the at least one of the one or more elongated expansion elements between a delivery configuration extending along the longitudinal axis and an expanded configuration extending laterally away from the longitudinal axis to shift the expandable section between a delivery configuration and an expanded configuration.

In some aspects, the expansion section is mounted over a shaft of the cutting device proximal to a cutting edge of the cutting device.

In some aspects, the expansion section is mounted over a shaft defining a lumen therethrough through which the cutting device is axially translatable.

In some aspects, the expandable section is operably associated with a shaft of a cutting device.

In some aspects, the system further includes a proximal support along a proximal end of the expandable section, and a distal support along a distal end of the expandable section, and the one or more elongated expansion elements include a plurality of elongated expansion elements extending longitudinally along and circumferentially around the expandable section. In some aspects, each of the plurality of elongated expansion elements has a distal end coupled to the distal support and a proximal end coupled to the proximal support. In some aspects, at least one of the distal support or the proximal support is segmented circumferentially to define support segments which move longitudinally separately and independently of one another to shift the elongated expansion elements separately and independently of one another between the delivery configuration and the expanded configuration thereof.

In accordance with various principles of the present disclosure, a method of moving anatomical tissue includes creating an incision using a cutting device; inserting the distal end of an expansion section of a tissue-moving device into the incision, the expansion section including a plurality of elongate expansion elements each having a proximal end and a distal end; and shifting one or more of the elongate expansion elements of the expansion section from a delivery configuration to an expanded configuration by moving together the proximal end and the distal end of each of the one or more elongate expansion elements along the expansion section to further separate tissue at the incision.

In some aspects, the method further includes expanding at least one elongate expansion element separately and independently of others of the elongate expansion elements.

In some aspects, one of the proximal end or the distal end of the at least one elongate expansion element is coupled to a support element segment movable toward the other of the proximal end or the distal end of the at least one elongate expansion element, and the method further includes moving the support element segment with respect to the others of the elongate expansion elements to expand the at least one elongate expansion element separately and independently of others of the elongate expansion elements.

In some aspects, the distal end of each of the elongate expansion elements is coupled to a distal support along the distal end of the expansion section, and the proximal end of each of the elongate expansion elements is coupled to a proximal support along the proximal end of the expansion section, and at least one of the distal support or the proximal support is segmented circumferentially to define support segments which move longitudinally separately and independently of one another, the method further comprising shifting the segments to shift elongated expansion elements separately and independently of one another between the delivery configuration and the expanded configuration thereof. In some aspects, at least two elongate expansion elements are coupled to each support segment, further comprising moving elongate expansion elements coupled to a first support segment independently and separately from elongate expansion elements coupled to a second support segment.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue-moving device and system formed in accordance with aspects of the present disclosure.

FIG. 2 illustrates a perspective view of a device and system as illustrated in FIG. 1 with an expandable section of the tissue-moving device expanded.

FIG. 3 illustrates a side elevational view of the device and system of FIG. 2 positioned in a schematic representation of tissue being separated by the device and system.

FIG. 4 illustrates an end view of the device and system illustrated in FIG. 3.

FIG. 5 illustrates a perspective view of a device and system as illustrated in FIG. 2 with an additional expandable section expanded.

FIG. 6 illustrates a side elevational view of the device and system of FIG. 5 positioned in a schematic representation of tissue being separated by the device and system.

FIG. 7 illustrates an end view of the device and system illustrated in FIG. 6.

FIG. 8 illustrates a perspective view of a device and system as illustrated in FIG. 5 with an additional expandable section expanded.

FIG. 9 illustrates a side elevational view of the device and system of FIG. 8 positioned in a schematic representation of tissue being separated by the device and system.

FIG. 10 illustrates an end view of the device and system illustrated in FIG. 9.

FIG. 11 illustrates a view of an example of an embodiment of a device and system as illustrated in FIG. 1 with a modified expandable section engaging tissue of a schematic representation of tissue being separated by the device and system.

FIG. 12 illustrates a perspective view of an example of an embodiment of a tissue-moving device similar to that of FIG. 1, but with a modified expandable section.

FIG. 13 illustrates a side elevational view of the device of FIG. 12 positioned in a schematic representation of tissue being separated by the device and system.

FIG. 14 illustrates a perspective view of an alternative example of an embodiment of a tissue-moving device and system formed in accordance with various principles of the present disclosure.

FIG. 15 illustrates a perspective view of a device and system as illustrated in FIG. 14 with an expandable section of the tissue-moving device expanded.

FIG. 16A illustrates a perspective view of an alternative example of an embodiment of a device and system formed in accordance with various principles of the present disclosure.

FIG. 16B illustrates a perspective view of a device and system as illustrated in FIG. 16A with an expandable section of the tissue-moving device expanded.

FIG. 16C illustrates a perspective view of a device and system as illustrated in FIG. 16B with an instrument further advanced distally with respect to the tissue-moving device.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during positioning or delivery), and/or closest to a delivery device, and “distal” refers to the direction or location furthest from the user, such as when using the device (e.g., introducing the device into a patient, or during positioning or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, a device configured to move, such as to separate and/or lift and/or apply traction to an anatomical structure. The anatomical structure may be anatomical tissue, and the device may be configured to move a layer of tissue relative to another layer of tissue. It will be appreciated that reference may be made herein simply to “tissue” instead of “anatomical structure” for the sake of convenience and without intent to limit. The device includes an expandable section which may be considered to form a scaffold which may move or lift an anatomical structure, such as tissue, with respect to another anatomical structure, such as tissue from which the lifted tissue is separated. It will be appreciated that the device is referenced herein as a “tissue-moving device” for the sake of convenience and without intent to limit. In some aspects, the tissue-moving device is mounted with respect to another medical instrument. It will be appreciated that reference may be made interchangeably herein to terms such as a medical instrument, tool, device, accessory, etc., without intent to limit. The tissue-moving device may be operated to move tissue with respect to (e.g., away from) the medical instrument, such as before, after, and/or during a procedure performed using the medical. In some aspects, the tissue-moving device is mounted alongside and/or over the medical instrument. In some aspects, a portion of the tissue-moving device circumferentially surrounds a portion of the medical instrument. In some aspects, the medical instrument is a cutting device and the tissue-moving device separates and/or applies traction to tissue cut by the cutting device, such as to move and/or lift away the tissue from underlying tissue. In some aspects, the medical instrument is movable (e.g., advanceable and/or retractable and/or rotatable) independently of the tissue-moving device.

In some aspects, the use of a tissue-moving device formed in accordance with various principles of the present disclosure may facilitate tissue separation by presenting a greater surface area to separate tissue than a typical cutting edge. In some aspects, the tissue-moving device has an expandable section which may be considered to form a scaffold for supporting and/or moving tissue. In some aspects, the tissue-moving device separates adjacent layers of tissue, such as different types of tissue. For instance, the tissue-moving device may be used to separate submucosal tissue from muscularis tissue during a third space endoscopy procedure. In some aspects, the tissue-moving device may be formed with a relatively blunt configuration, generally rounded edges without sharp or acute edges, and/or with generally rounded edges, and does not present a risk of inadvertently cutting tissue or other structures not intended to be cut.

It will be appreciated that a tissue-moving device formed in accordance with various principles of the present disclosure may be used in performing a procedure using open surgery (accessing the interior of a patient's body by cutting open the body) or minimally invasive surgery (e.g., percutaneously, laparoscopically, endoscopically, etc.). In some aspects, both the tissue-moving device and the cutting device are configured to be extended through a working channel of a medical scope and advanced to a target site within a patient. In some aspects, the tissue-moving device is expandable from a compact delivery configuration (e.g., capable of being extended through a delivery device, such as a working channel of a medical scope) to an expanded configuration (such as with a greater cross-sectional dimension). In some aspects, the tissue-moving device includes one or more expansion elements configured and arranged to shift from a generally elongated compact configuration (e.g., along the cutting device, such as generally parallel to a shaft of the cutting device) to an expanded configuration extending radially outwardly from the cutting device. The expansion elements may be flexible elongate elements which may, in some aspects, be considered scaffold elements. It will be appreciated that terms such as expanded, bowed, flexed, bent, etc., (including other grammatical forms of such terms) may be usable interchangeably herein to refer to a configuration in which the expansion elements of the tissue-moving device extends radially away from the longitudinal axis of the tissue-moving device and/or system. In such configuration, the tissue-moving device may separate tissue, such as to move tissue away from a tissue layer being cut by the cutting device and/or to separate layers of tissue from each other.

Various embodiments of tissue-moving devices and associated systems and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified herein by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers with a ′ added, and redundant description is generally omitted for the sake of brevity. Moreover, certain features in one embodiment may be used across different embodiments and are not necessarily individually labeled when appearing in different embodiments.

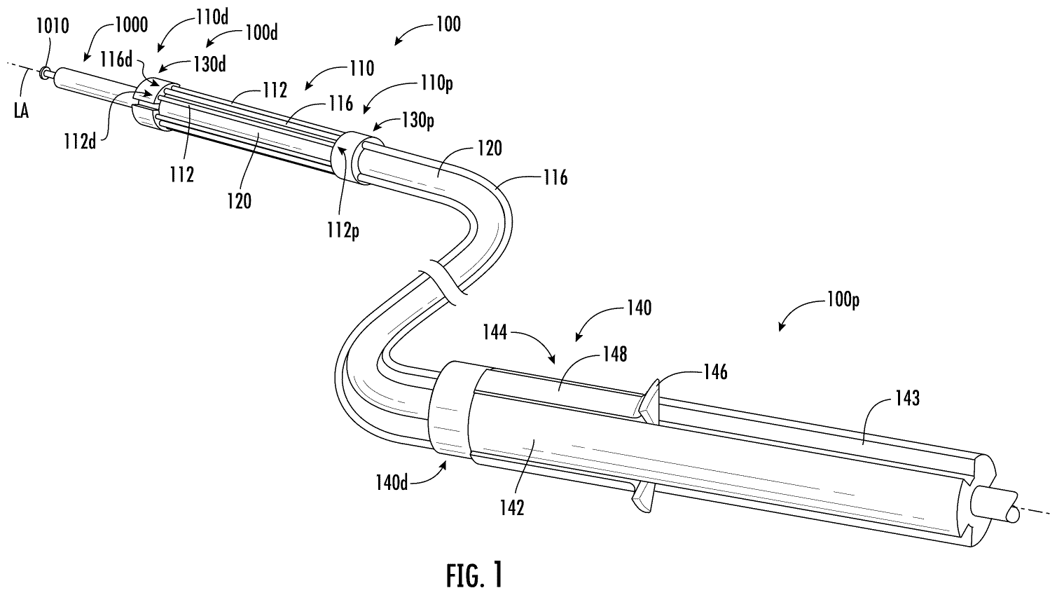

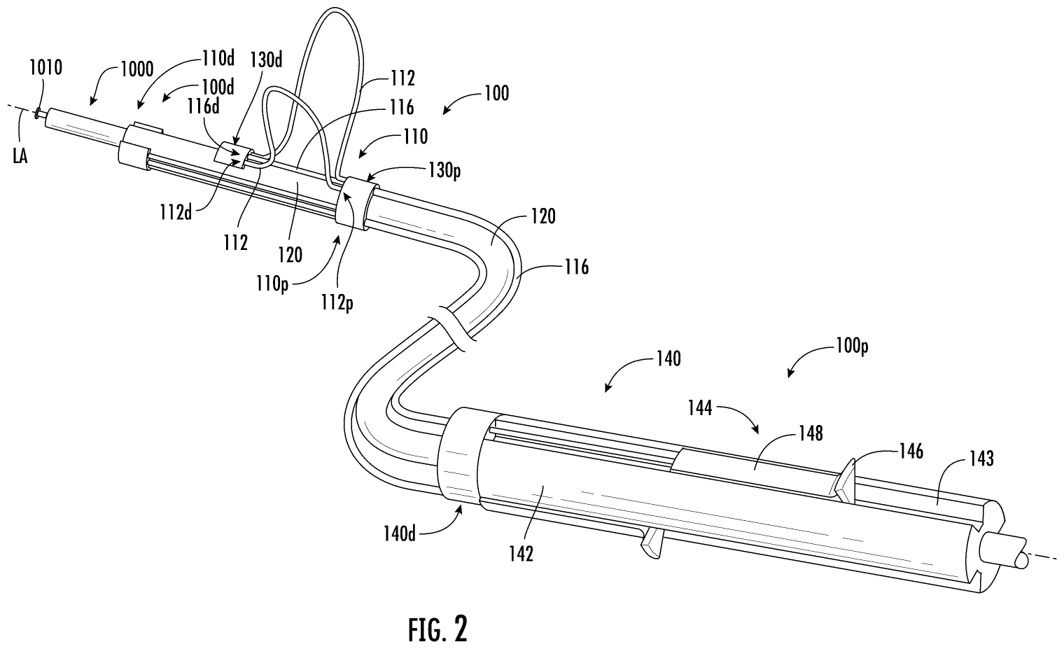

Turning now to the drawings, a tissue-moving device 100 formed in accordance with various principles of the present disclosure is illustrated in FIG. 1 in a delivery configuration, and in FIG. 2 in an expanded configuration. More particularly, the distal end 100d of the tissue-moving device 100 includes an expandable section 110 configured to expand from the delivery configuration illustrated in FIG. 1 to the expanded configuration illustrated in FIG. 2. In some aspects, the tissue-moving device 100 is operably associated with, such as mounted or coupled with respect to, a shaft 120 of another medical accessory, or a shaft 120 defining a lumen therethrough, through which another medical accessory may be movably extended, as described in further detail below. It will be appreciated that the shaft 120 may be an elongate flexible member capable of being navigated within a patient's body (such as through tortuous passages within the patient's body). In some aspects, the shaft 120 is sized, shaped, configured, and/or dimensioned to extend though a working channel of a medical scope (e.g., endoscope), such as known to those of ordinary skill in the art and thus not illustrated. Expansion of the expandable section 110 (e.g., a portion of the expandable section 110, as discussed in greater detail below) of the tissue-moving device 100 may be laterally with respect to the longitudinal axis LA of the shaft, such as radially away from the longitudinal axis LA. As may be appreciated, for the sake of convenience, and without intent to limit, reference is made to delivery and expanded configurations of the tissue-moving device 100 generally, even if the different configurations are along the expandable section 110, without significant differences in configuration along portions of the tissue-moving device 100 proximal or distal to the expandable section 110. As also may be appreciated, the delivery configuration is a generally compact configuration facilitating delivery of the tissue-moving device 100 within a patient's body (e.g., transluminally, endoluminally, etc., such as through tortuous pathways within the patient's body, such as through a working channel of a medical scope or other delivery device). The tissue-moving device 100 thus is preferably sufficiently flexible for such delivery. Moreover, it will be appreciated that a dimension, such as (but not necessarily limited to) a cross-sectional area, of the tissue-moving device 100 is increased upon shifting the tissue-moving device 100 from the delivery configuration to the expanded configuration.

In the example of an embodiment of a tissue-moving device 100 illustrated in FIG. 1, the expandable section 110 is formed of one or more elongated expansion elements 112 which allow various configurations and orientations during a medical procedure to achieve the desired effect on tissue, such as to facilitate the procedure. In some aspects, two or more elongated expansion elements 112 are used, spaced apart from one another to provide an area therebetween and/or to form a scaffold for engaging and moving tissue. In some aspects, the elongated expansion elements 112 of the expandable section 110 are each formed of a flexible material capable of shifting from a generally elongated configuration (such as illustrated in FIG. 1) to a bowed configuration (such as illustrated in FIG. 2). For instance, the elongated expansion elements 112 may be formed of a metal or polymeric material or combination thereof, suitable for medical use and preferably resistant to kinking (such as when navigated through tortuous body passages) and with deformations under the elastic limit. Such materials include, without limitation, medical grade stainless steel, nitinol, polyetheretherketone (PEEK), a thermoplastic elastomer (e.g., a polyether block amide such as PEBAX®, sold by Arkema Global), polytetrafluoroethylene (PTFE), etc., the present disclosure not being limited in this regard. The elongated expansion elements 112 are preferably sufficiently flexible to bow into an expanded configuration when actuated at the proximal end 100p of the tissue-moving device 100 (as discussed in further detail below). It will be appreciated, however, that the elongated expansion elements 112 should be sufficiently stiff to be able to move tissue upon expansion of the elongated expansion elements 112. However, it should also be appreciated that, in some aspects, the cross-sectional shape, material, thickness, stiffness, and other relevant properties are selected so that the elongated expansion elements 112 do not cut through tissue upon expanding. For instance, in some aspects, the elongated expansion elements 112 are formed to have blunt, and no sharp edge. In some aspects, the elongated expansion elements 112 extend longitudinally along and circumferentially around the longitudinal axis LA of the tissue-moving device 100 and expand laterally/radially outwardly from the longitudinal axis LA (e.g., from a generally elongated, generally straight configuration, substantially parallel to the longitudinal axis LA) to increase a cross-sectional area defined by the expandable section 110 of the tissue-moving device 100.

In use, the distal end 100d of the tissue-moving device 100 is advanceable, when in the delivery configuration, into an anatomical structure, such as tissue, and expandable to move portions of the anatomical structure with respect to other portions of the anatomical structure, such as illustrated in FIGS. 3-10. For instance, the distal end 100d of the tissue-moving device 100, when in a delivery configuration, may be inserted into tissue to move the tissue upon expansion of the expandable section 110. In some aspects, the tissue-moving device 100 presents a surface (e.g., a distal end) which is relatively blunt and not configured to cut tissue, and is used in conjunction with the sharper cutting device 1000 having a cutting edge 1010 capable of cutting anatomical structures, such as a blade, knife, electrocautery knife, tissue resecting tool, needle, laser, scissors, etc., such as may be selected based on the particular procedure to be performed (the present disclosure not being limited in this regard). In some aspects, the distal end 100d of the tissue-moving device 100 may be inserted into an incision made in tissue by the cutting edge 1010 of the cutting device 1000, and then shifted to an expanded configuration such as to further separate tissue layers initially separated by the incision.

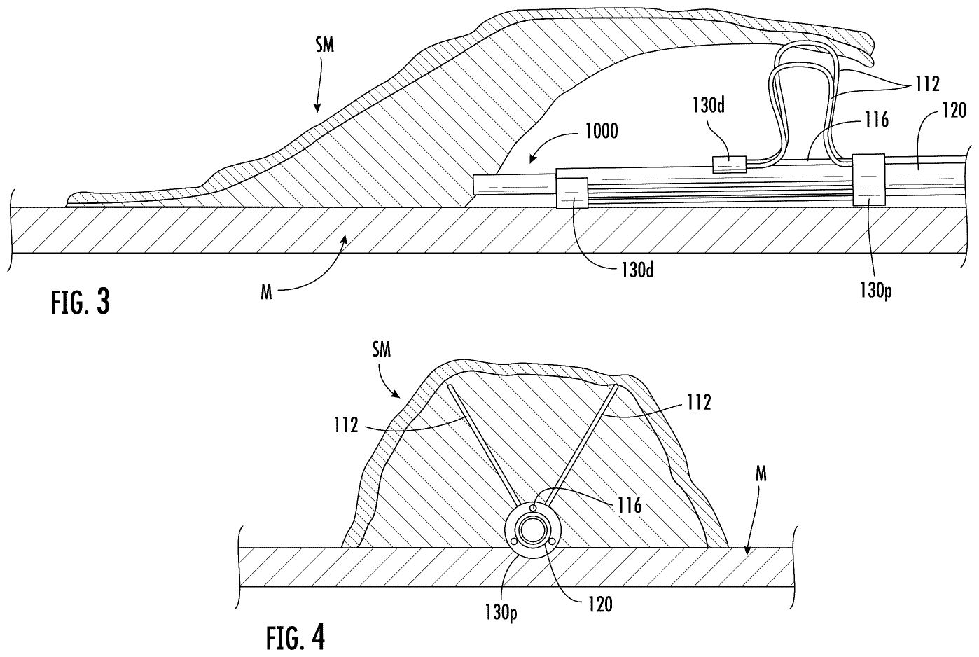

The expandable section 110 of a tissue-moving device 100 formed in accordance with various principles of the present disclosure is configured to improve tissue separation over what is achievable with use of only a cutting device 1000. In some aspects, a tissue-moving device 100 formed in accordance with various principles of the present disclosure is configured to improve control of tissue separation over control achievable by prior devices. For instance, in accordance with various principles of the present disclosure, the one or more elongated expansion elements 112 may be actuated individually or in groups to achieve different expansion configurations. In some aspects, at least two elongated expansion elements 112 are expanded together to define at least two spaced-apart regions of contact with tissue to move (e.g., lift) a section of tissue extending between the at least two elongated expansion elements 112, such as illustrated in FIG. 3 and FIG. 4. In some aspects, the elongated expansion elements 112 may be considered to form a scaffold for the tissue. Such scaffold may facilitate lifting of a section of tissue more efficiently than if lifted by a single elongated expansion element 112. In some aspects, if two elongated expansion elements 112 are expanded together, with other elongated expansion elements 112 (if present) unexpanded, the tissue-moving device 100 may be used to move tissue directionally, i.e., in the direction of expansion of the expanded elongated expansion elements 112. Such directionality may allow for selective and/or controlled moving of tissue in a selected direction by rotating the expandable section 110 to direct the expanded elongated expansion elements 112 towards tissue to be moved.

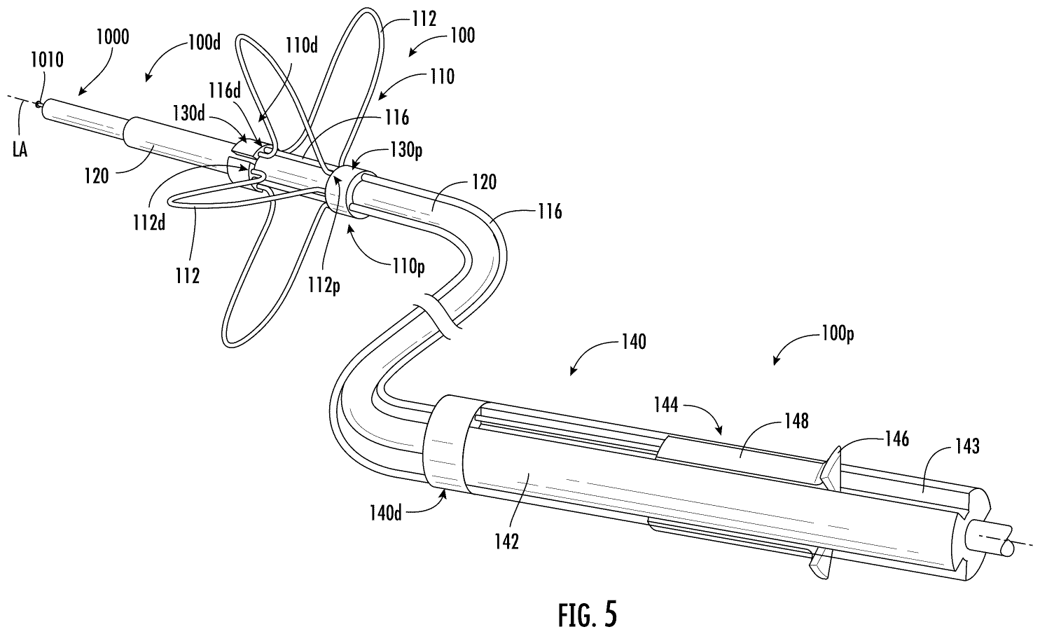

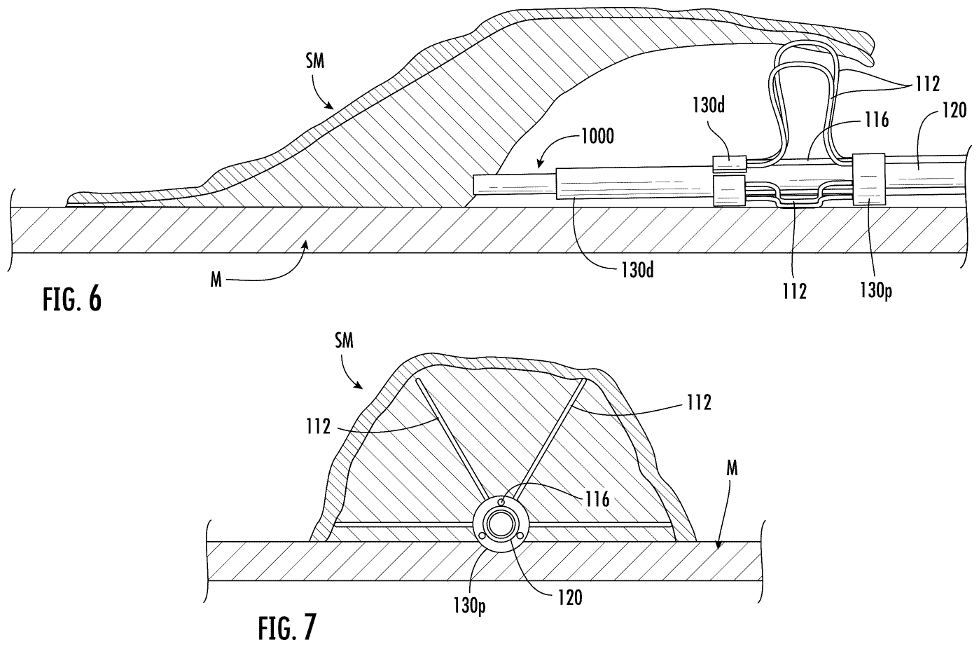

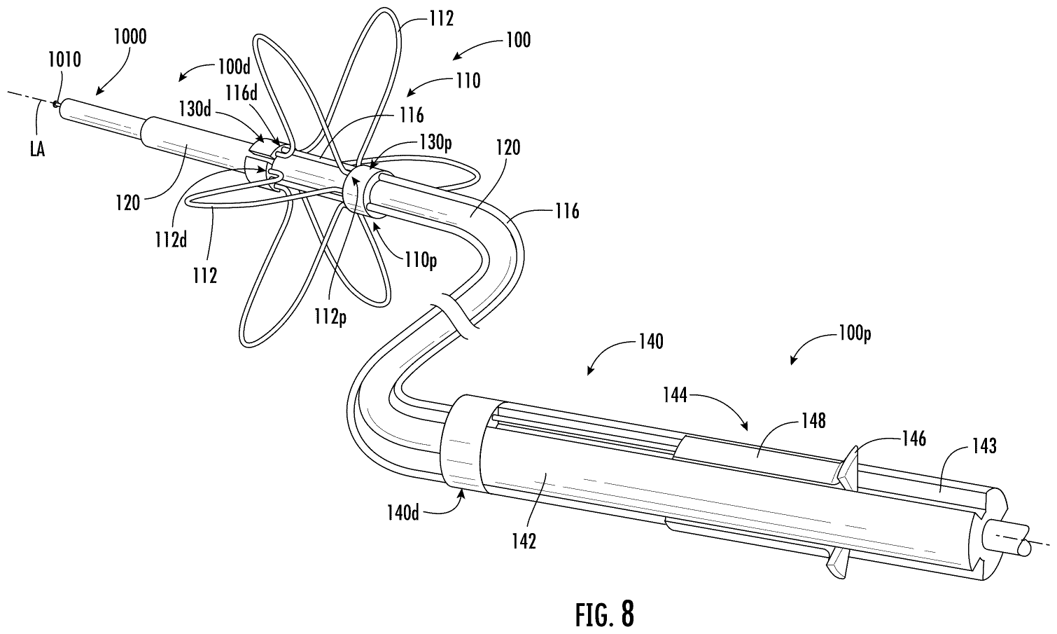

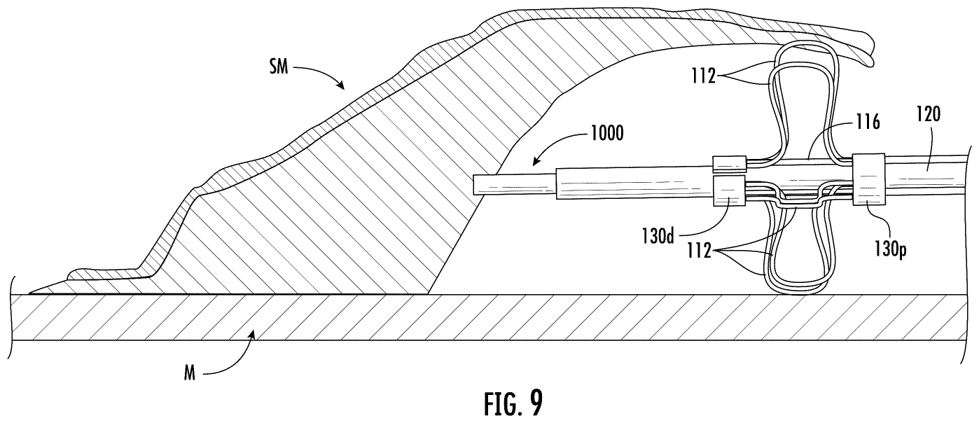

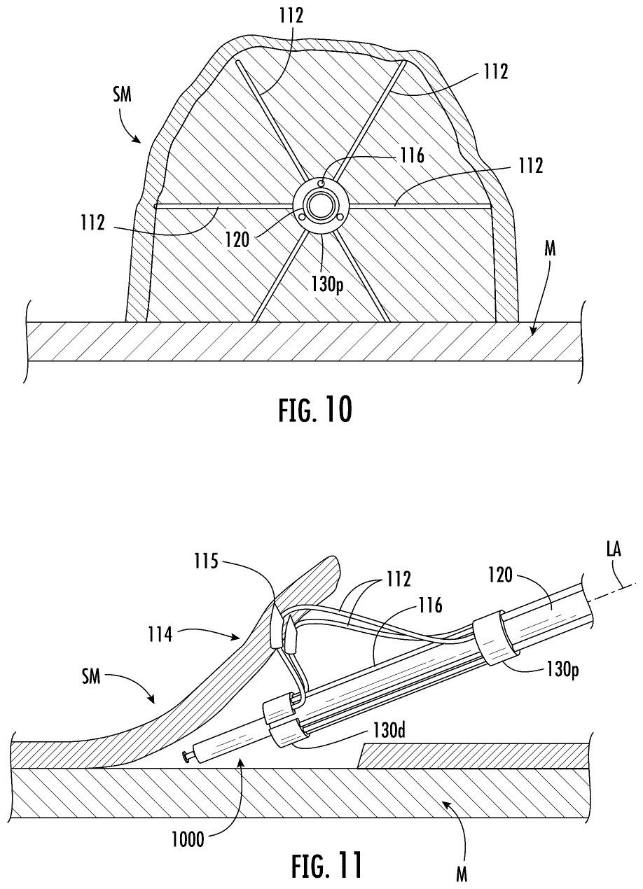

It will be appreciated that a tissue-moving device 100 formed in accordance with various principles of the present disclosure may allow a variety of configurations of expansion of the elongated expansion elements 112 to be achieved to move tissue as desired, indicated, needed, etc., during a procedure. Instead of just a pair of elongated expansion elements 112 being expanded as in FIG. 3 and FIG. 4, two adjacent pairs of elongated expansion elements 112 may be expanded, as illustrated in FIG. 5, FIG. 6, and FIG. 7, to move a greater area of tissue. Additionally or alternatively, elongated expansion elements 112 extending in generally opposite directions may be expanded to increase the radial expansion of the expandable section 110 (e.g., increase the cross-sectional area of the expandable section 110), such as illustrated in FIG. 8, FIG. 9, and FIG. 10. As may be appreciated, an increased radial expansion of the expandable section 110 increases the effect of the tissue-moving device 100 on tissue.

As may be appreciated, various combinations of expansion of the elongated expansion elements 112 may be used during a procedure for separating submucosal tissue from muscularis tissue. For instance, submucosal tissue SM may be initially separated from muscularis tissue M by a cutting device 1000, and the distal end 100d of the tissue-moving device 100 inserted thereafter. At least one or two elongated expansion elements 112 may then be expanded, such as illustrated in FIG. 2, FIG. 3, and FIG. 4 to initiate separation of the submucosal tissue SM and the muscularis tissue M (as illustrated in FIG. 3 and FIG. 4). The tissue-moving device 100 may be advanced further between the submucosal tissue SM and the muscularis tissue M to separate the tissues SM and M. Additional elongated expansion elements 112 may be expanded alongside the already expanded elongated expansion elements 112 to increase the area of tissue being moved by the tissue-moving device 100, as illustrated in FIG. 5, FIG. 6, FIG. 7, FIG. 8, FIG. 9, and FIG. 10. Additionally or alternatively, the elongated expansion elements 112 may be expanded in different directions from the direction in which the already expanded elongated expansion elements 112 extend to increase the distance tissue is being moved by the tissue-moving device 100. Various combinations and/or degrees of expansion may be used without limit.

In accordance with various principles of the present disclosure, the elongated expansion elements 112 may be actuated in any of a variety of manners to shift from a delivery configuration to an expanded configuration. To achieve a variety of different expansion configurations as described above, the elongated expansion elements 112 may be actuated separately from one another (such as individually or in groups of two or more, but not all, of the elongated expansion elements 112 of the expandable section 110). In some aspects, a proximal end 112p of each elongated expansion element 112 is coupled to a proximal end 110p of the expandable section 110, and a distal end 112d of each elongated expansion element 112 is coupled to a distal end 110d of the expandable section 110. In some aspects, the ends 112p, 112d of the elongated expansion elements 112 are moved with respect to each other to shift the elongated expansion elements 112 between delivery (e.g., extended, generally elongated, substantially straight, etc.) and expanded (e.g., bowed, bent, moved radially outwardly, etc.) configurations.

In the example of an embodiment illustrated in FIG. 1 and FIG. 2, a proximal end 112p of each elongated expansion element 112 is coupled to a proximal support 130p along a proximal end 110p of the expandable section 110, and a distal end 112d of each elongated expansion element 112 is coupled to a distal support 130d along a distal end 110d of the expandable section 110. Relative movement of the distal support 130d and the proximal support 130p causes the one or more elongated expansion elements 112 coupled thereto to shift between a delivery configuration and an expanded configuration. For instance, movement of the distal support 130d and the proximal support 130p towards each other may cause expansion of the elongated expansion elements 112, and movement of the distal support 130d and the proximal support 130p away from each other may cause extension of the elongated expansion elements 112 into a more compact configuration, defining a smaller overall cross-sectional area than when in the expanded configuration. In some aspects, the distal support 130d and the proximal support 130p are formed as generally tubular or annular elements defining a passage therethrough. The above-described shaft 120 may extend through the supports 130 to mount or otherwise operably associate the expandable section 110 with the shaft 120.

To achieve separate, individual, independent actuation of the elongated expansion elements 112, at least one of the distal support 130d or the proximal support 130p may be segmented into more than one support element separately shiftable independently of the other support elements. For instance, groups of one or more elongated expansion elements 112 of the tissue-moving device 100 may be actuatable to move in a different manner from the remaining elongated expansion elements 112 of the elongated expansion elements 112 by being coupled with a separately and independently movable support 130d and/or 130p. In the example illustrated in FIG. 1, the distal support 130d is segmented. However, it will be appreciated that the present disclosure is not limited to this example, and other configurations are within the scope and spirit of the present disclosure. As may be appreciated, segmenting of one or both of the supports 130 allows movement of the one or more elongated expansion elements 112 coupled to only the support 130 which is being moved. As such, the elongated expansion elements 112 coupled to the support 130 being shifted may be shifted as well, with the support 130, separately and independently of other elongated expansion elements 112 which need not be shifted at the same time and which are not coupled to the support 130 which is being shifted. In the illustrated embodiments, the distal support 130d is segmented in circumferential increments (around the circumference thereof). The circumferential increments may be evenly spaced (e.g., to create uniform spacing of groups of elongated expansion elements 112 which may be actuated independently of one another) or spaced at different increments, the present disclosure not being limited in this regard. In some aspects, the segments of a support 130 are operably associated with an underlying structure (e.g., a circumferentially extending base element of the support 130 and/or with the shaft 120 extending through the support) to remain in place laterally with respect to the longitudinal axis LA of the expandable section 110 (i.e., not to move laterally or radially away from the other segments of the support 130). For instance, a projection or wedge or other engagement element on the segment may engage a slot or slit or other corresponding engagement element holding the segment 130 in place with respect to the longitudinal axis LA. Additionally or alternatively, segments of a support 130 may not move laterally outwardly away from the longitudinal axis LA due to the tension in the segments when the elongated expansion elements 112 are extended and/or stretched into a generally straight configuration. It will be appreciated the present disclosure need not be limited to circumferentially defined segments, and other configurations allowing for separate and independent movement may be used instead without detracting from the present disclosure.

As noted above, in order to shift an elongated expansion element 112 between a delivery configuration and an expanded configuration, the proximal end 112p and the distal end 112d of the elongated expansion element 112 are brought closer together (to expand) or further apart (e.g., to elongate the elongated expansion element 112 to a delivery configuration). In some aspects, each end 112p, 112d of an elongated expansion element 112 is a free, terminal end thereof. Accordingly, in such embodiment, neither end 112p, 112d is accessible for control at the proximal end 100p of the tissue-moving device 100. In the example of an embodiment of a tissue-moving device 100 illustrated in FIGS. 1-13, a control element 116 is operably associated with the elongated expansion elements 112 to shift the elongated expansion elements 112 between the delivery configuration and the expanded configuration. The illustrated control element 116 is in the form of an elongate control element 116 extending proximally to the proximal end 100p of the tissue-moving device 100 for access, control, actuation, etc., at the proximal end 100p by a user. In some aspects, the elongate control element 116 is sufficiently elongated and flexible to be delivered within a patient's body (e.g., sufficiently flexible to be navigated transluminally within a patient's body) with the tissue-moving device 100. In some aspects, the elongate control element 116 has sufficient pull strength to pull the elongated expansion elements 112 to actuate the elongated expansion elements 112. In some aspects, the elongate control element 116 is a wire (e.g., a control wire), a cord, a string, a strand, a filament, a rod, a stylet, etc., the present disclosure not being limited in this regard. As may be appreciated, the elongate control element 116 may be operably associated with one or more elongated expansion elements 112. In the illustrated embodiment, in which a pair of elongated expansion elements 112 are actuatable together, the elongate control element 116 is operably associated with a pair of elongated expansion elements 112. It will be appreciated that other configurations are within the scope and spirit of the present disclosure, the present disclosure not being limited to a particular number of elongated expansion elements 112 which shift together or a particular number of elongated expansion elements 112 controlled by a given/particular elongate control element 116.

In some aspects, an elongate control element 116 is operably associated with a support 130 with which one or more elongated expansion elements 112 are operably associated. In the illustrated examples of embodiments, an elongate control element 116 is coupled to the distal support 130d. However, an elongate control element 116 may instead be coupled to the proximal support 130p, the present disclosure not being limited in this regard. Movement of the elongate control element 116 causes the support 130 with which the elongate control element 116 is coupled to move, thereby shifting the configuration of the elongated expansion elements 112 associated therewith. In the example of an embodiment illustrated in FIGS. 1-10, the elongate control element 116 is operably associated with a segment of a support 130 with which one or more elongated expansion elements 112 are operably associated. In some aspects, each segment of a support 130 of the expandable section 110 may have an associated elongate control element 116 to be controlled separately and independently of the other segments of the support 130. The various configurations of the elongated expansion elements 112 of the tissue-moving device 100 illustrated in FIGS. 1-10 illustrate separate and independent movement of the illustrated elongate control elements 116 relative to one another to shift different elongated expansion elements 112 relative to one another to achieve different configurations as medically indicated, desired, needed, etc., such as during a procedure in which the tissue-moving device 100 is being used.

The elongate control element 116 may be controlled in any of a variety of manners. In the example of an embodiment illustrated in FIG. 1, FIG. 2, FIG. 5, and FIG. 8, the elongate control element 116 extends proximally to a control handle 140 operable by a user (e.g., a medical professional) to actuate the elongate control element 116 to control shifting of an elongated expansion element 112 associated therewith. In accordance with various principles of the present disclosure, each elongate control element 116 is separately and independently controlled to separately and independently control one or more elongated expansion elements 112 operably associated therewith. In the illustrated example of an embodiment, the control handle 140 includes a main body 142 (which may alternately be referenced as a plunger, body, shaft, elongate member, housing, frame, etc., without intent to limit) and one or more actuators 144 (which may alternately be referenced as a controller, slider, movable user-engagement element, etc., without intent to limit) operably associated with the main body 142. Each elongate control element 116 is operably associated with an actuator 144 to move upon movement of the actuator 144 to shift the configuration of the associated one or more elongated expansion elements 112 (operably associated with the control element 116). The actuator 144 may include a user engagement feature 146, such as a radially-outwardly extending tab or other feature differentiated from adjacent features of the actuator 144 to facilitate engagement of a user's finger with the actuator 144 to move the actuator 144 to actuate a selected elongate control element 116. In some aspects, an actuator 144 and an elongate control element 116 are operably associated to axially translate the elongate control element 116 and a support 130 with respect to the shaft 120 to thereby shift the configuration of elongated expansion elements 112 operably associated therewith. In some aspects, the proximal support 130p is mounted on the shaft 120, such as fixedly mounted, and the proximal end 120p of the shaft 120 is operably associated with (e.g., coupled to) the distal end 140d of the control handle 140. In such embodiment, the actuator 144 and the elongate control element 116 may be operably associated with the distal support 130d. Axial translation of the actuator 144 with respect to the control handle 140 thus axially translates the actuator 144 and the distal support 130d with respect to the shaft 120 and the proximal support 130p, thereby moving the distal end 112d and the proximal end 112p of each of the associated one or more elongated expansion elements 112 with respect to each other to shift the associated one or more elongated expansion elements 112 between delivery and expanded configurations. In some aspects, the actuator 144 allows independent control and shifting of the elongated expansion elements 112 with which the actuator 144 is operably associated, to different extents than the other elongated expansion elements 112 are shifted. For instance, as described above, selected elongated expansion elements 112 are shiftable by selective movement of an associated actuator 144 while others of the elongated expansion elements 112 are not shifted. Additionally or alternatively, the actuators 144 need not shift the elongated expansion elements 112 to the same extent. For instance, the elongated expansion elements 112 need not be shifted to a fully expanded configuration, but, instead, may be shifted to a partially expanded configuration instead.

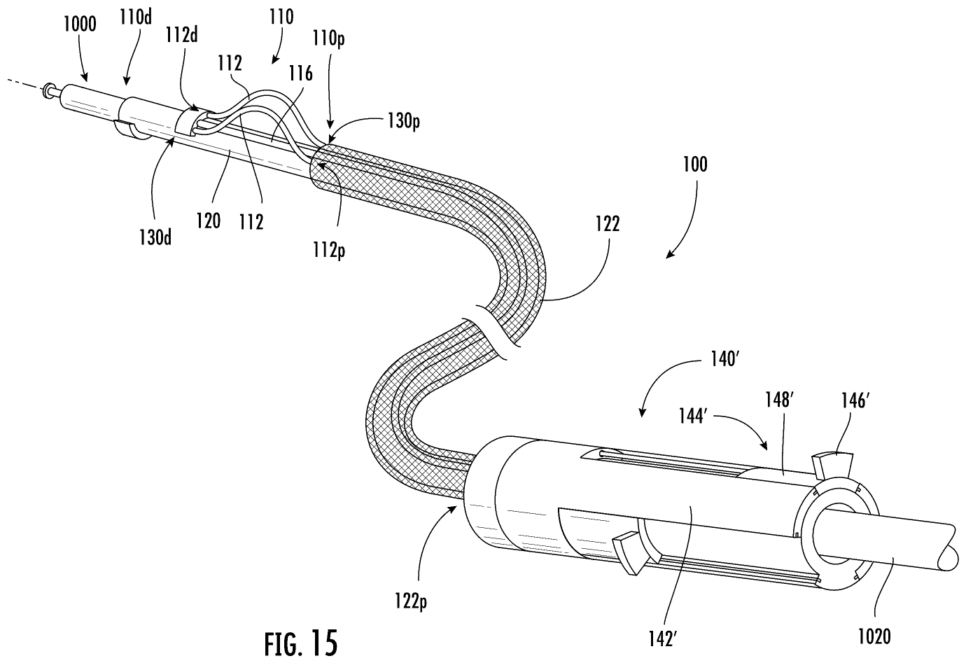

In some aspects, the main body 142 may include a groove or channel 143 in which an actuator 144 may be operably associated with the main body 142 and in which the actuator 144 is movable. The actuator 144 may be configured to fit within and with respect to a channel 143 to axially translate therein to advance or retract the associated elongate control element 116 to actuate an associated elongated expansion element 112. In some aspects, the actuator 144 and channel 143 are sized, shaped, configured, and/or dimensioned to be held with respect to each other so that the actuator 144 is coupled with respect to the main body 142. For instance, the user engagement feature 146 and the channel 143 may have complementary shapes (e.g., complementary tapered or wedge shapes, complementary semicircular shapes, complementary slot shapes, etc.) which hold the actuator 144 in place with respect to the channel 143 and thus with respect to the main body 142. Additionally or alternatively, in an alternative example of an embodiment of control handle 140′ illustrated in FIG. 14 and FIG. 15, the actuator 144′ may have a base element 148′ and channel 143′ with complementary shapes (e.g., a matching tongue and groove configuration). It will be appreciated that the main body 142′ and/or the user engagement feature 148′ of the actuator 144′ illustrated in FIG. 14 and FIG. 15 may be substantially the same as the main body 142 and user engagement feature 148 of the actuator 144 illustrated in FIG. 1 and FIG. 2, or may have other variations such as to size, shape, configuration, dimensions, etc. In some aspects, an independent user engagement feature 148 may be provided for each actuator 144. Other configurations known to those of ordinary skill in the art may be used, the present disclosure not being limited in this regard. It will be appreciated that although the illustrated example of an embodiment of an actuator 144 is axially slidable, other configurations and movement of actuators (e.g., rotatable knobs, etc.) are within the scope of the present disclosure.

It will be appreciated that various modifications may be made to various components of a tissue-moving device 100 formed in accordance with various principles of the present disclosure without departing from the spirit and scope of the present disclosure. For instance, various modifications may be made to the elongated expansion elements 112 of a tissue-moving device 100 formed in accordance with various principles of the present disclosure. In some aspects, one or more of the elongated expansion elements 112 may be configured to engage the tissue being moved by the elongated expansion elements 112 in a manner which maintains traction with the tissue. For instance, in the example of an embodiment illustrated in FIG. 11, one or more of the elongated expansion elements 112 may include a tissue anchor feature 114 configured to engage and to hold an elongated expansion element 112 with respect to tissue. In some aspects, each elongated expansion element 112 of at least a pair of elongated expansion elements 112 includes a tissue anchor feature 114 having a sharp tissue engaging tip 115 configured to penetrate tissue without damaging the tissue to hold the elongated expansion elements 112 with respect to the tissue and thereby maintain traction with respect to the tissue as the elongated expansion element 112 is expanded. Although the tissue anchor feature 114 is illustrated as an element which may be formed separately from the elongated expansion elements 112, it will be appreciated that other configurations are within the scope and spirit of the present disclosure, such as, without limitation, tissue engaging tips formed integrally with (as a part of) the elongated expansion element 112 and which extend away from the remaining proximal and distal portions of the elongated expansion element 112 as the elongated expansion element 112 expands. Typically, the tissue anchor feature 114 is positioned along a radially outermost extending portion of the elongated expansion element 112 on which the sharp tissue engaging tip 115 is positioned. Additionally or alternatively, the sharp tissue engaging tip 115 is directed proximally, as illustrated in FIG. 11. However, other configurations, orientations, positions, etc., of the sharp tissue engaging tip 115 are within the scope of the present disclosure, the present disclosure not being limited by the illustrated embodiment.

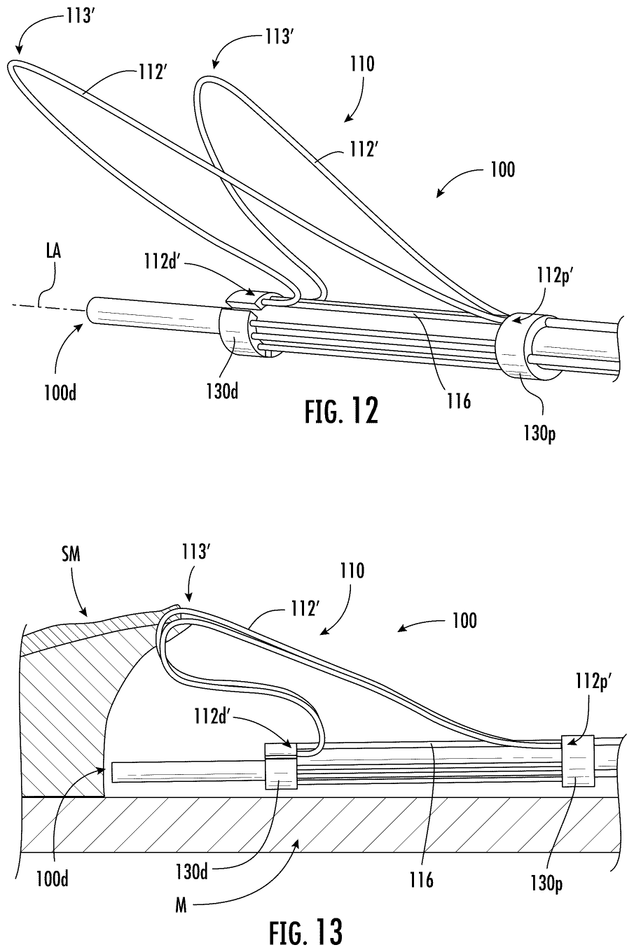

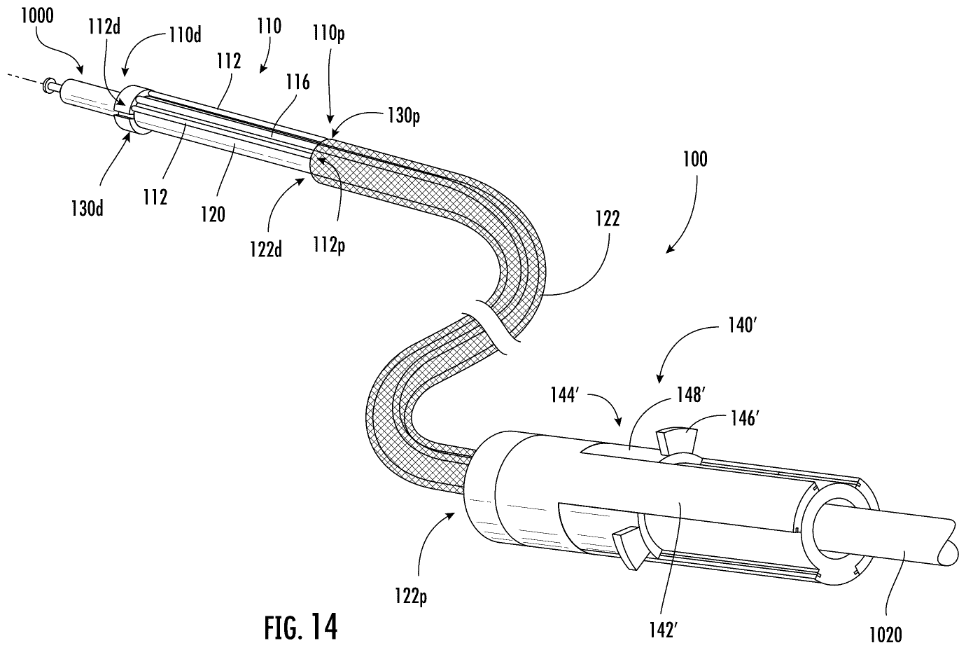

In some aspects, the expanded configuration of one or more of the elongated expansion elements 112 may be preset or otherwise predefined to achieve a desired shape to enhance moving, shifting, lifting, etc., of tissue. In the example of an embodiment illustrated in FIG. 12 and FIG. 13, one or more, and preferably two or more elongated expansion elements 112′ are formed to expand into a configuration selected not only to lift tissue radially away from the expandable section 110 (transversely with respect to the longitudinal axis LA) but also to push tissue distally. More particularly, the elongated expansion elements 112′ are configured to expand into a shape which is biased distally. As the elongated expansion elements 112′ expand, the radially-outermost portion 113′ of the elongated expansion element 112′ (furthest from the longitudinal axis LA of the tissue-moving device 100) is further from the proximal end 112p′ of the elongated expansion element 112′than from the distal end 112d′ of the elongated expansion element 112′. In some aspects, the radially-outermost portion 113′ of the elongated expansion elements 112′ (in a direction transverse to the longitudinal axis LA) extends distally past the distal end 112d′ of the elongated expansion element 112′. In some aspects, the radially-outermost portion 113′ of the elongated expansion element 112′ extends distally past the distal end 100d of the tissue-moving device 100. As an elongated expansion element 112′ shifts from a delivery configuration to an expanded configuration, the radially-outermost portion 113′ moves radially away from the longitudinal axis LA of the tissue-moving device 100 as well as distally, moving tissue radially outwardly and distally as well.

It will be appreciated that various other modifications may be made to various other components of a tissue-moving device 100 formed in accordance with various principles of the present disclosure without departing from the spirit and scope of the present disclosure. For instance, in some aspects, at least a portion of the shaft of a tissue-moving device 100 formed in accordance with various principles of the present disclosure may be in the form of a braided shaft 122, such as illustrated in FIG. 14 and FIG. 15. As may be appreciated, a braided shaft 122 may be useful for facilitating navigation and/or torquing of the tissue-moving device 100 to advance the expandable section 110 to tissue to be separated. The braided shaft 122 may be formed by braiding, intertwining, or otherwise interengaging a plurality of filaments, such as wires, fibers, etc., such as in a manner known to those of ordinary skill in the art. The cross-section of the filaments may be round, square, rectangular, flat, etc. The braided filaments may be formed of any suitable material such as, without limitation, stainless steel. In some aspects, the braided filaments may be encased, coated, covered, embedded, etc., in another material such as a polymeric material. In some aspects, the braided shaft has a polymeric material (e.g., a thermoplastic elastomer such as PEBAX®) reflowed over the braid to insulate the braid. Optionally, an additional tubular member can be attached over the braided shaft. A cutting device 1000 may translatably extend through a lumen defined longitudinally through the braided shaft 122′, such as illustrated in FIG. 14. In some aspects, the proximal support 130p may be operably associated with the braided shaft 122. For instance, the proximal support 130p may be formed as a part of the braided shaft 122. In such configuration, the proximal support 130p may be considered to be a distal portion of the braided shaft 122. Additionally or alternatively, at least a portion of the elongated expansion elements 112 may be formed as extensions of the braided shaft 122 (e.g., as filaments of the braided shaft extending beyond a distal end 120d of the braided shaft 122 and not braided, and/or otherwise extending generally axially). In some aspects, the proximal ends 112p of the elongated expansion elements 112 may be directly coupled to the braided shaft 120′, such as, without limitation, by reflow of the material of the elongated expansion elements 112 (e.g., if formed of a polymeric material), welding, brazing, adhering, and/or using an additional material such as heat shrink material, tape, etc., or other coupling technique known to those of ordinary skill in the art. It will be appreciated that various other components of the tissue-moving device 100 illustrated in FIG. 14 and FIG. 15 (other than the control handle 140, as described above) are similar to the components described above with reference to FIGS. 1-13, reference being made to the above descriptions thereof for the sake of brevity and without intent to limit.

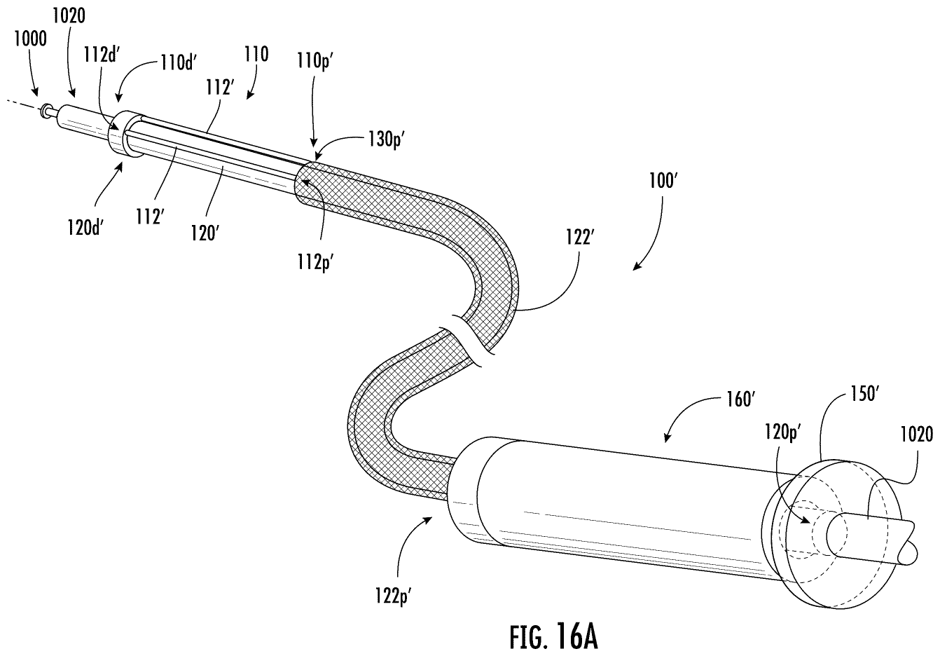

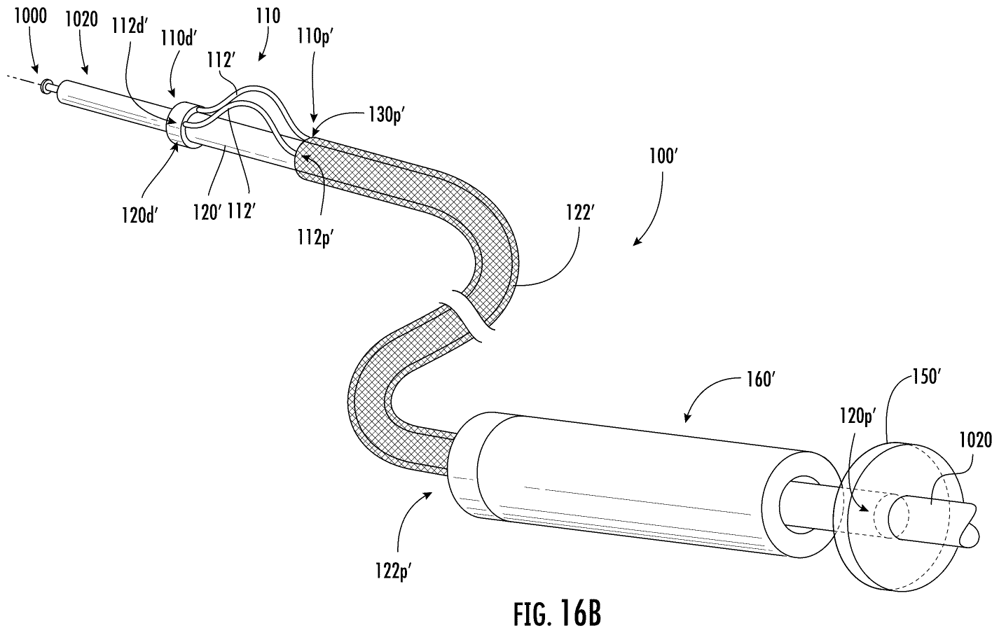

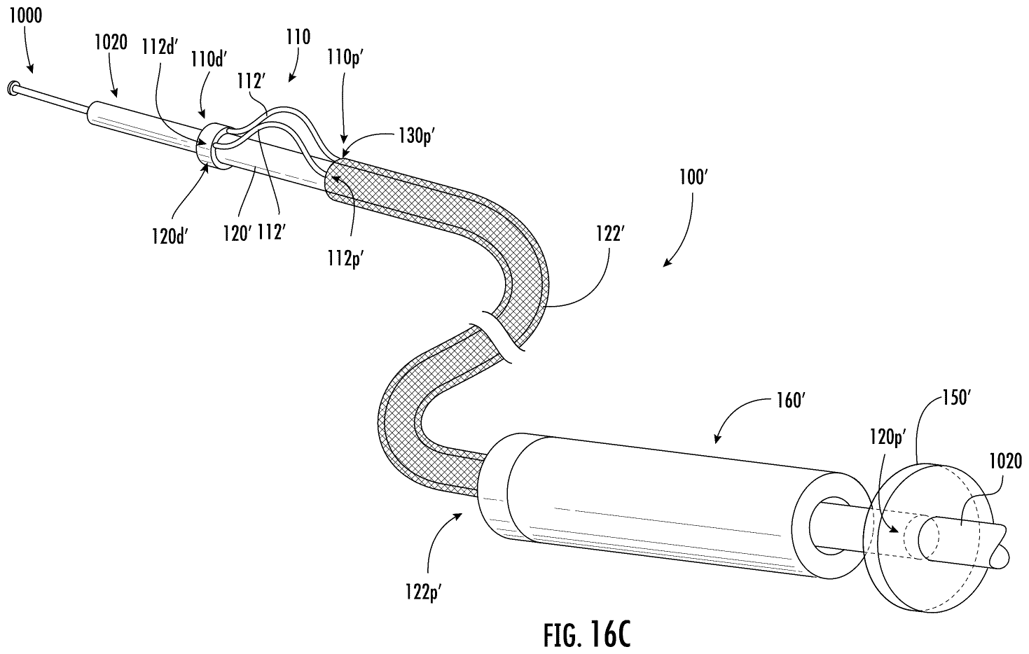

In some aspects, a shaft 120′ is extended through a lumen defined through a braided shaft 122′, such as in the example of an embodiment of a tissue-moving device 100′ illustrated in FIG. 16A, FIG. 16B, and FIG. 16C. The proximal end 120p′ of the shaft 120′ is operably associated with a shaft control handle 150′ and the proximal end 122p′ of the braided shaft 122′ is operably associated with a separate and independently controllable braided shaft handle 152′ The shaft 120′ is independently longitudinally translatable with respect to the braided shaft 122′ to shift the elongated expansion elements 112′ of the expandable tissue-moving device 100 between delivery and expanded configurations, such as may be appreciated upon comparison of FIG. 16A and FIG. 16B. More particularly, in the example of an embodiment illustrated in FIG. 16A, FIG. 16B, and FIG. 16C, the shaft 120′ is axially movable through the braided shaft 122′ and axially movable over the shaft 1020 of the cutting device 1000 (which may extend through or along the shaft 120′). In FIG. 16A, the expandable section 110′ is in a delivery configuration with the distal support 130d′ separated from the proximal support 130p0′ (which is illustrated as formed along and/or as a part of the distal end 122d′ of the braided shaft 122′). In FIG. 16B, the control handle 150′ is moved proximally to move the shaft 120′ proximally.

Proximal movement of the shaft 120′ moves the distal support 130d′ closer to the proximal support 130p′ to shift the expandable section 110′ to an expanded configuration. In some aspects, the proximal support 130p′ is formed similar to the manner in which the proximal support 130p described above with reference to FIG. 14 and FIG. 15 is formed, reference being made to the above description with respect to the proximal support 130p as applicable to the proximal support 130p′, without intent to limit. In some aspects, such as illustrated in FIG. 16C, the cutting device 1000 is movable with respect to the shaft 120′ and the braided shaft 122′. The cutting device 1000 may thus be moved distally (as shown, and/or proximally) with respect to the expandable section 110′ (operably coupled with the shaft 120′ and the braided shaft 122′). For instance, the shaft 1020 of the cutting device 1000 (extending proximal to the control handle 150′ in the illustrated example of an embodiment) may be axially translated relative to the shaft control handle 150′ and the braided shaft control handle 160′. The cutting device 1000 may thereby be operated in a position spaced distally from the expandable section 110 if desired or needed, with the expandable section 110′ remaining expanded, thus maintaining separation of tissue as desired or needed. It will be appreciated that a similar cutting device handle and similar arrangement may be provided in the tissue-moving device 100 illustrated in FIGS. 1-10 if provided with a shaft 120 separate and independent of the cutting device 1000.

Although embodiments of the present disclosure may be described with specific reference to third space endoscopy and/or separation of mucosal and muscularis tissue, it is appreciated that various other procedures may similarly benefit from the structures and methods disclosed herein. It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A medical device configured to move tissue, said medical device comprising:
an expandable section having a proximal end and a distal end and extending along a longitudinal axis of said medical device; and
one or more elongated expansion elements each having a proximal end coupled with respect to the proximal end of said expandable section and a distal end coupled with respect to the distal end of said expandable section;
wherein the proximal end and distal end of at least one of said one or more elongated expansion elements are movable with respect to each other to shift said at least one of said one or more elongated expansion elements between a delivery configuration extending along the longitudinal axis and an expanded configuration extending laterally away from the longitudinal axis to shift said expandable section between a delivery configuration and an expanded configuration.

2. The medical device of claim 1, further comprising:
a proximal support along a proximal end of said expandable section, said proximal end of said at least one of said one or more elongated expansion elements coupled to said proximal support; and
a distal support along a distal end of said expandable section, said distal end of said at least one of said one or more elongated expansion elements coupled to said distal support;
wherein said proximal support and said distal support are movable with respect to each other to shift said at least one of said one or more elongated expansion elements between the delivery configuration and the expanded configuration.

3. The medical device of claim 1, wherein said one or more elongated expansion elements comprise a plurality of elongated expansion elements extending longitudinally along and circumferentially around said expandable section.

4. The medical device of claim 3, wherein:
each of said plurality of elongated expansion elements has a distal end coupled to said distal support and a proximal end coupled to said proximal support; and
at least one of said distal support or said proximal support is segmented circumferentially to define support segments which move longitudinally separately and independently of one another to shift the elongated expansion elements separately and independently of one another between the delivery configuration and the expanded configuration thereof.

5. The medical device of claim 2, further comprising a control element operably coupled with one of said distal support or said proximal support to move said proximal support and said distal support with respect to each other.

6. The medical device of claim 5, wherein said control element has a proximal end extending proximal to the proximal end of said at least one of said one or more elongated expansion elements and to a control handle at a proximal end of said medical device.

7. The medical device of claim 6, wherein one of said proximal support or said distal support is annular defining a passage therethrough and movable with respect to a shaft extended through the passage.

8. The medical device of claim 7, wherein the other of said proximal support or said distal support is fixed with respect to the shaft.

9. The medical device of claim 8, wherein the shaft defines a lumen therethrough through which a cutting device is axially translatable with respect to said expandable section.

10. The medical device of claim 1, wherein said expandable structure is operably associated with a shaft defining a lumen therethrough through which a cutting device is axially translatable with respect to said expandable section.

11. A system for separating tissue, said system comprising:
16. a tissue moving device having an expandable section having a proximal end and a distal end and extending along a longitudinal axis of said medical device, and one or more elongated expansion elements each having a proximal end coupled with respect to the proximal end of said expandable section and a distal end coupled with respect to the distal end of said expandable section; and
17. a cutting device, said expandable section operably associated with said cutting device;
18. wherein the proximal end and distal end of at least one of said one or more elongated expansion elements are movable with respect to each other to shift said at least one of said one or more elongated expansion elements between a delivery configuration extending along the longitudinal axis and an expanded configuration extending laterally away from the longitudinal axis to shift said expandable section between a delivery configuration and an expanded configuration.

12. The system of claim 11, wherein said expansion section is mounted over a shaft of said cutting device proximal to a cutting edge of said cutting device.

13. The system of claim 11, wherein said expansion section is mounted over a shaft defining a lumen therethrough through which said cutting device is axially translatable.

14. The system of claim 11, wherein said expandable section is operably associated with a shaft of a cutting device.

15. The system of claim 11, further comprising a proximal support along a proximal end of said expandable section, and a distal support along a distal end of said expandable section, wherein:
19. said one or more elongated expansion elements comprise a plurality of elongated expansion elements extending longitudinally along and circumferentially around said expandable section;
20. each of said plurality of elongated expansion elements has a distal end coupled to said distal support and a proximal end coupled to said proximal support; and
21. at least one of said distal support or said proximal support is segmented circumferentially to define support segments which move longitudinally separately and independently of one another to shift the elongated expansion elements separately and independently of one another between the delivery configuration and the expanded configuration thereof.

16. A method of moving anatomical tissue, said method comprising:
22. creating an incision using a cutting device;
23. inserting the distal end of an expansion section of a tissue-moving device into the incision, the expansion section including a plurality of elongate expansion elements each having a proximal end and a distal end; and
24. shifting one or more of the elongate expansion elements of the expansion section from a delivery configuration to an expanded configuration by moving together the proximal end and the distal end of each of the one or more elongate expansion elements along the expansion section to further separate tissue at the incision.

17. The method of claim 16, further comprising expanding at least one elongate expansion element separately and independently of others of the elongate expansion elements.

18. The method of claim 17, wherein one of the proximal end or the distal end of the at least one elongate expansion element is coupled to a support element segment movable toward the other of the proximal end or the distal end of the at least one elongate expansion element, said method further comprising moving the support element segment with respect to the others of the elongate expansion elements to expand the at least one elongate expansion element separately and independently of others of the elongate expansion elements.

19. The method of claim 16, wherein the distal end of each of the elongate expansion elements is coupled to a distal support along the distal end of the expansion section, and the proximal end of each of the elongate expansion elements is coupled to a proximal support along the proximal end of the expansion section, and at least one of the distal support or the proximal support is segmented circumferentially to define support segments which move longitudinally separately and independently of one another, said method further comprising shifting the segments to shift elongated expansion elements separately and independently of one another between the delivery configuration and the expanded configuration thereof.

20. The method of claim 19, wherein at least two elongate expansion elements are coupled to each support segment, further comprising moving elongate expansion elements coupled to a first support segment independently and separately from elongate expansion elements coupled to a second support segment.
